(19) United States
(12) Patent Application Publication
(10) Pub. No.: US 2010/0260787 A1
(43) Pub. Date: Oct. 14, 2010

(54) Human sperm fibrous sheath (FS) proteins: new target antigens for use in therapeutic cancer vaccines and diagnostic screening and Methods of Using Same

(75) Inventors: Maurizio Chiriva Internati, Lubbock, TX (US); Everardo Cobos, Lubbock, TX (US); Wijbe Martin Kast, Los Angeles, CA (US); Kenneth Morrow, JR., New Port, KY (US)

Correspondence Address: Maurizio Chiriva Internati, 5202 26th St. Rear, Lubbock, TX, 79407, US

(73) Assignee: Kiromic Inc., Lubbock, TX (US)

(21) Appl. No.: 12/421,356

(22) Filed: Apr. 9, 2009

(51) Int. Cl. A61K 39/00; C07K 14/00; A61K 38/00

(52) U.S. Cl. 424/184.1; 530/350; 514/12

(57) ABSTRACT

Cancer vaccines were demonstrated to be promising strategies for cancer treatment but the strategies are limited by the paucity of target antigens that provoke an effective immune response. We propose that sperm fibrous sheet proteins constitute a new class of potential antigens for cancer vaccines. This hypothesis is supported by the expression of the sperm fibrous sheath protein known as Sperm protein 17 detected in tumors of unrelated histological origin. It has the ability to induce T-cell based immune responses. The expression of the Sperm protein 17 (Sp17) in tumors of unrelated histological origin, and its natural localization in the human sperm fibrous sheath (FS), led us to hypothesize that FS proteins might represent a new potential class of target antigens useful for developing cancer vaccines and diagnostic and therapeutic treatments.

The human sperm fibrous sheath (FS): The flagellum of the mammalian spermatozoa consists of four distinct segments; a) the connecting piece adjacent to the head; b) the middle piece defined by a tightly packed helical array of mitochondria surrounding the cytoskeletal structures of the flagellum; c) the principal piece, and d) the short end piece.

The major cytoskeletal structures are the axoneme, also present in cilia, and the outer dense fibers and FS, which are unique to spermatozoa. The FS is a unique cytoskeletal entity, which underlies the plasma membrane, surrounds the axoneme and outer dense fibers, and defines the extent of the principal piece of the sperm flagellum [1]. it consists of two longitudinal columns connected by closely arranged semicircular ribs that assemble from distal to proximal throughout spermatogenesis [1,2]. Although the function of FS is unclear, it is believed to serve as a scaffold for both glycolytic enzymes and constituents of the signaling cascades, and it is well positioned to play a role in the regulation of sperm motility [1]. Several proteins localized in the FS were identified, including Sp17, CABYR, AKAP-3, AKAP-4, TAKAP-80, Rhopilin, Ropporin, GSTM5. There are no doubts numerous others molecules belongs to the FS and yet to be discovered. Of these Sp17 and CABYR were thoroughly analyzed:

Sperm protein 17(Sp17): A family of tumor-associated antigens, called cancer-testis (CT) antigens was found in a limited number of normal human tissues and various human tumors of unrelated histological origin [2]. One of these, Sp17, was identified as a CT antigen in multiple myeloma, other blood malignancies, and ovarian cancer. An mRNA encoding Sp17 was detected in 17% of patients with multiple myeloma and in the primary tumor cells from 70% of patients with primary ovarian carcinoma [3,4]. At the protein level, Sp17 was found in human germinal cells of the testis (except in the case of spermatogonia) [5], the ciliated epithelia of the respiratory airways, and both the male and female reproductive systems [6]. It was recently found in the synoviocytes of females affected by rheumatoid arthritis [7] and the melanophages of cutaneous melanocytic lesions [8], as well as in a proportion of primary nervous system tumors [9] and a subset of esthesioneuroblastomas [10]. As it is expressed in germinal cells and various neoplastic tissues, Sp17 is more widely distributed in humans than originally thought. Although the function of Sp17 is still unknown, the high degree of, sequence conservation throughout its N-terminal half, and the presence of an A-kinase anchoring protein (AKAP)-binding motif within this region, suggests that it might play a regulatory role in a protein kinase A (PKA)-independent AKAP complex in both germinal and neoplastic cells.

Conclusions. Since the first cloning of a human tumor antigen [11], the identification and development of immunogenic cancer vaccines targeting these antigens represents a formidable task [12, 13, 14, 15]. We hypothesize that the sperm FS protein, for example, Sp17 constitutes a new class of target antigens for developing cancer vaccines. The present hypothesis will increase the number of available target antigens in cancer vaccines and for the development of therapeutics and diagnostic treatments [2, 16, 17].

### REFERENCES

[1] Eddy E M, Toshimori K, O'Brien D A. FS of mammalian spermatozoa; Microsc Res Tech 2003; 61: 103-115.

[2] Simpson A J, Caballero O L, Jungbluth A, Chen Y T, Old L J. Cancer/testis antigens, gametogenesis and cancer. Nat Rev Cancer. 2005: 5: 615-625.

[3] Lim S H, Wang Z, Chiriva-Internati M, Xue Y. Sperm protein 17 is a novel cancer-testis antigen in multiple myeloma. Blood. 2001; 97: 1508-1510.

[4] Chiriva-Internati M, Wang Z. Salati E, Timmins P, Lim S H. Tumor vaccine for ovarian carcinoma targeting sperm protein 17. Cancer. 2002; 94: 2447-2453.

[5] Grizzi F. Chiriva-Internati M, Franceschini B, Hermonat P L, Soda G, Lim S H, Dioguardi N. Immunolocalization of sperm protein 17 in human testis and ejaculated spermatozoa. J Histochem Cytochem. 2003; 51: 1245-1248.

[6] Grizzi F. Chiriva-Internati M, Franceschini B, Bumm K, Colombo P, Ciccarelli M, Donetti E, Gagliano N. Hermonat P L, Bright R K, Gioia M, Dioguardi N, Kast W M. Sperm protein 17 is expressed in human somatic ciliated epithelia. J Histochem Cytochem. 2004; 52: 549-554.

[7] Takeoka Y, Kenny T P, Yago H, Naiki M, Gershwin M E, Robbins D L. Developmental considerations of sperm protein 17 gene expression in rheumatoid arthritis synoviocytes. Dev Immunol. 2002; 9: 97-102.

[8] Franceschini B, Grizzi F, Colombo P, Soda G, Bumm K, Hermonat P L, Monti M, Dioguardi N, Chiriva-Internati M. Expression of human sperm protein 17 in melanophages of cutaneous melanocytic lesions. Br J Dermatol. 2004; 150: 780-782.

[9] Grizzi F, Gaetani P, Franceschini B, Di leva A, Colombo P, Ceva-Grimaldi G, Bollati A, Frezza E E, Cobos E, Rodriguez y Baena R, Dioguardi N, Chiriva-Intemati M. Sperm protein 17 is expressed in human nervous system tumors. BMC Cancer. 2006; 6: 23.

[10] Bumm K, Grizzi F, Franceschini B, Koch M, Iro H, Wurm J, Ceva-Grimaidi G, Dimmler A, Cobos E, Dioguardi N, Sinha U K, Kast W M, Chiriva-Internati M. Sperm protein 17 expression defines 2 subsets of primary esthesioneuroblastoma. Hum Pathol. 2005; 36:1289-1293.

[11] van der Bruggen P, Traversari C, Chomez P, Lurquin C, De Plaen F, Van den Eynde B, Knuth A, Boon T. A gene encoding an antigen recognized by cytolytic T lymphocytes on a human melanoma. Science 1991; 254: 1643-1647

[12] Gilboa E. The promise of cancer vaccines. Nat Rev Cancer 2004; 4: 401-411.

[13] Brinkman J A, Hughes S H, Stone P, Caffrey A S, Muderspach L I, Roman L D, Weber J S, Kast W M. Therapeutic vaccination for HPV induced cervical cancers. Disease Markers 2007; 23:1-16.

[14] Berzofsky J A, Terabe M, Oh S, Belyakov I M, Ahlers J D, Janik J E, Morris J O. Progress on new vaccine strategies for the immunotherapy and prevention of cancer. J Clin Invest. 2004; 113: 1515-1525.

[15] Chiriva-Internati M, Grizzi F, Bright R K, Kast W M. Cancer immunotherapy: avoiding the road to perdition. J Transl Med. 2004; 2: 26.

[16] Costa F F, Le Blanc K, Brodin B. Concise review: cancer/testis antigens, stem cells, and cancer. Stem Cells. 2007; 25: 707-711.

[17] Kalejs M, Erenpreisa J. Cancer/testis antigens and gametogenesis: a review and “brain-storming” session. Cancer Cell Int. 2005; 5: 4

1. The discovery of new molecules from the FS proteins might represent a new potential class of target antigens. These antigens can be useful for developing cancer vaccines and diagnostic and therapeutic treatments (in several formulations such as: genes, proteins, antibodies, and DNA). The FS antigens are potentially useful for developing cancer vaccines or diagnostic kits for cancer prevention and therapeutic applications.

2. The discovery of the first anatomical structure FS will be used as a protein and DNA to induce a vaccine pulsing in dendritic cells or using just the single FS as a structure that can stimulate protection against this class of antigens that are expressed in different tumors such as ovarian, lung, prostate, breast, colon, pancreas cancers and multiple myeloma, liver, brain, stomach, cervical, anal, as well as other hematological and non-hematological tumors. It will be used to activate the immune system in in vivo and ex vivo applications. These FS molecules can be used for potential drug discovery and development.

3. As claimed in claim 1 the development of novel reagents such as different antigens, genes, proteins, DNA, antibodies from the FS structures will be analyzed by microarray. New FS antigens can be discovered by micro-array analysis.

4. As claimed in claim 1 the FS proteins can constitute a potential group of new cancer testis antigens for different application in the health system.

5. As claimed in claim 1 the use of the fibrous sheath (genes and proteins and antibodies derived from the fibrous sheath structure), an anatomical structure, used as a protein or DNA can stimulate anti-tumor immunity.

6. As claimed in claim 1 the novelty is the anatomical structure to be used for the first time as a protein, genes, DNA, and antibodies to stimulate anti-tumor immunity.

7. As claimed in claim 1 any cancer-related antigens, proteins genes and antibodies derived from the FS, for example (Sperm Protein 17, AKAP-4, ASP, CABIR, PTTG-1, ROPPORIN) and all others potential proteins, genes or antibodies that can potentially be derived from a FS component which will be used to develop diagnostic kits for cancer prevention.

8. As claimed in claim 1 any cancer-related antigen, proteins genes or antibodies derived from the fibrous sheath, for example (Sperm Protein 17, AKAP-4, ASP, CABIR, PTTG-1, and ROPPORIN), which will be used to develop a therapeutic cancer vaccine.

9. As claimed in claim 2 any cancer-related antigens, proteins genes and antibodies derived from the fibrous sheath, for example Sperm Protein 17, AKAP-4, ASP, CABIR, PTTG-1, ROPPORIN, will be used to develop a diagnostic test for cancer prevention. Furthermore, it will be used for potential drug discovery and development for each protein, gene, DNA, antibody development from the FS structure.

10. As claimed in claim 2 any cancer-related antigens, proteins genes and antibodies derived from the fibrous sheath, for example antibodies directed against the Sperm Protein 17, AKAP-4, ASP, CABIR, PTTG-1, ROPPORIN to be used in therapeutic and diagnostic applications.
